(19) United States
(12) Patent Application Publication
(10) Pub. No.: US 2019/0274631 A1
(43) Pub. Date: Sep. 12, 2019

(54) SYSTEM AND METHOD FOR MONITORING PHYSIOLOGICAL PARAMETERS WITHIN LIVING BODIES

(71) Applicant: Integrated Sensing Systems, Inc., Ypsilanti, MI (US)

(72) Inventor: Nader Najafi, Ann Arbor, MI (US)

(21) Appl. No.: 16/293,193

(22) Filed: Mar. 5, 2019

Related U.S. Application Data
(63) Continuation of application No. 62/710,928, filed on Mar. 5, 2018, now abandoned.

(51) Int. Cl. A61B 5/00; G16H 50/20; G16H 40/67; G16H 80/00; G06N 20/00; A61B 5/0215

(52) U.S. Cl. CPC A61B 5/7267; G16H 50/20; G16H 40/67; G16H 80/00; A61B 5/7282; A61B 5/0215; A61B 5/0022; A61B 5/747; A61B 5/4839; G06N 20/00

(57) ABSTRACT

Methods and systems for managing at least one chronic disease in a population of patients by collecting information from the population of patients and prescribing courses of medical action based on collaboration between human beings and artificial intelligence (AI). Sensors are placed within the human bodies of the population of patients to continuously sense and monitor at least one physiological parameter and generate data corresponding to the physiological parameter, and the data are transmitted via the Internet and stored in a database. Artificial intelligence is then used to prescribe a course of medical action in an individual patient if the data indicates a medical condition that is within the database and known by the artificial intelligence. The artificial intelligence is also used to request an individualized course of medical action from a medical provider if the data indicates a medical condition not within the database or known by the artificial intelligence.

### CROSS-REFERENCE TO RELATED APPLICATIONS

This application claims the benefit of U.S. Provisional Application No. 62/710,928, filed Mar. 5, 2018, the contents of which are incorporated herein by reference.

### BACKGROUND OF THE INVENTION

The present invention generally relates to systems and methods for monitoring and treating large populations of individuals, as a nonlimiting example, individuals suffering from chronic diseases, such as heart failure (HF).

The world is facing serious challenges to manage millions of people with ubiquitous chronic diseases, particularly as a result of the average human age getting older and the percentage of older populations increasing. Aging naturally results in the development of common and ubiquitous diseases at unprecedented levels. Among the most important problems that many developed countries face is the affordable and effective management of chronic diseases. Current medical approaches are becoming obsolete and must be replaced, and new solutions are required to manage millions of people with chronic diseases. Among these epidemic-scale chronic diseases, perhaps the most important is heart failure (HF). Heart failure statistics in the U.S. include 6.5+ million Americans affected, 300,000+ new cases per year, and high morbidity (one in five HF patients die in the first year, and half of HF patients die within five years). One in nine deaths in 2009 in the U.S. was HF related.

Without new solutions, the cost of medical care for ubiquitous chronic diseases could soon bankrupt many government health agencies. As such, a new paradigm is needed to both effectively and affordably manage millions of people with chronic diseases. The effectiveness of medication-based treatments depends on regular titrations and adjustments of drugs customized for each patient over his/her lifetime. Also, it is becoming more evident that many medical device solutions require proper medication and sometimes adjustments for their long life operation, as an example, to optimize the benefits of ventricular assist devices (VAD), including left and right ventricular assist devices (LVADs and RVADs, respectively) that help the heart pump blood to the lungs and body.

### BRIEF SUMMARY OF THE INVENTION

The present invention provides systems and methods for monitoring and treating large populations of individuals, such as but not limited to individuals suffering from a chronic disease such as heart failure (HF).

According to one aspect of the invention, a method is provided for managing at least one chronic disease in a population of patients by collecting information from the population of patients and prescribing courses of medical action based on collaboration between human beings and artificial intelligence (AI). The method includes placing sensors within the human bodies of the population of patients to continuously or periodically sense and monitor at least one physiological parameter in the human bodies and generate data corresponding to the physiological parameter, and transmitting via the Internet the data corresponding to the physiological parameter in the population of patients and storing the data in a database. Artificial intelligence is then used to prescribe a course of medical action in an individual patient of the population of patients if the data corresponding to the physiological parameter in the patient indicates a medical condition that is within the database and known by the artificial intelligence. The artificial intelligence is also used to request an individualized course of medical action from a medical provider if the data corresponding to the physiological parameter in the patient indicates a medical condition not within the database or known by the artificial intelligence. The artificial intelligence then stores the medical condition and the individualized course of medical action prescribed by the medical provider in the database so that the artificial intelligence learns from the medical provider and the medical condition and the individualized course of medical action become known by the artificial intelligence.

Other aspects of the invention include systems adapted to perform steps as set forth above to monitor and prescribe individualized courses of medical action to treat individual patients of a population of patients.

Technical aspects of methods and systems as described above preferably include the ability to provide an effective and affordable treatment system to medically manage potentially millions of people with chronic diseases in general and with Heart Failure (HF) in particular. The preferred approach makes possible an intelligent, lifetime, individualized, and tailored treatment of each patient using a platform referred herein as Internet of Advanced Health, or IoAH.

Other aspects and advantages of this invention will be appreciated from the following detailed description.

### BRIEF DESCRIPTION OF THE SEVERAL VIEWS OF THE DRAWING

FIG. 1 contains a flow chart representing certain aspects of systems and procedures that utilize implantable sensing devices to monitor physiological parameters within large populations in accordance with nonlimiting embodiments of the invention.

### DETAILED DESCRIPTION OF THE INVENTION

The present invention encompasses the management of large-scale chronic diseases by collecting accurate information for a large population of patients, and using the Internet of Things (IoT) infrastructure to enable medical decisions to be made based on a collaboration between humans and artificial intelligence (AI). As it relates to heart diseases, the collected information includes data collected by sensors, for example, implantable hemodynamic monitor (IHM) sensors that have been chronically implanted in a large population of heart disease patients, to enable gathering of data and other accurate information over the lifetimes of the patients and then tailor and individualize treatments for each patient over his or her lifetime.

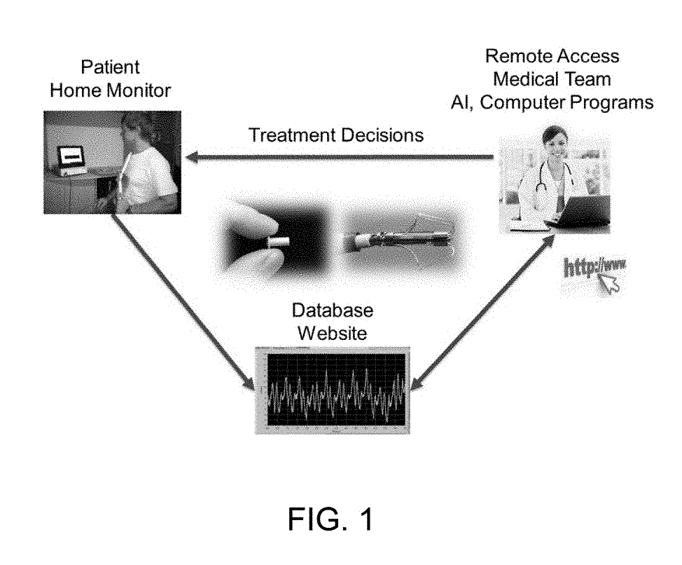

FIG. 1 contains a schematic diagram of a system and method for managing a very large population of heart failure patients that collects data regarding physiological parameters of the patients' hearts using IHM sensors chronically implanted within the cardiovascular systems of the patients, including but not limited to wireless IHM sensors implanted in their hearts or their pulmonary arteries. FIG. 1 represents an individual patient utilizing a reader unit to interrogate a wireless IHM sensor implanted in the patient's heart, and transmitting data obtained from the sensor to a monitor that displays a graph of a pressure within the patient's heart. The data are transmitted via the Internet to a database website that is remotely accessible by the members of the patient's medical team as well as an artificial intelligence capability (or other computer-based program) of the system. The medical team and artificial intelligence capability of the system are able to individually or collaboratively make medical decisions and prescribe courses of medical action for the patient in response to the data collected from the sensor. Two images located at the center of the diagram of FIG. 1 are representative of different modes by which the implantable IHM sensor can be anchored in the patient's heart.

Advances in artificial intelligence are enabling medical management and treatments smarter, more responsive, and better at making decisions in a variety of common conditions for which large amounts of data exist and are available. However, autonomous AI medical management systems are not adapted as yet to handle less common or unknown medical conditions and situations, or medical conditions and situations that are complicated or unpredictable. As such, FIG. 1 represents a method by which artificial intelligence (or other computer-based programs) can be used to provide medical management and treatments for relatively common conditions in heart disease patients and for which large amounts of data are available for autonomous processing by artificial intelligence, but then automatically seeks and requires human support to provide medical management and treatments for conditions for which sufficient data may not exist to permit the system to rely solely on artificial intelligence and for conditions and situations that are complicated or unpredictable. As such, the system depicted in FIG. 1 is a near autonomous system that is tended by humans. Overtime, the system may be able to collect sufficient data for conditions that at one point required human support so that artificial intelligence is able to provide management and treatments of patients with those same conditions without requiring human input or intervention. Such data may be collected by the system and/or medical researchers to gather Big Data from millions of patients.

In view of the above, the system is configured so that human intervention and artificial intelligence are both required and the system is not totally reliant on a fully automatic system, thereby overcoming major barriers to the safe and effective management and treatment of heart disease patients, while enabling the system to improve its capabilities over time in regard to conditions that are relatively uncommon, complicated, or unpredictable.

The system relies on the collection of data and information from a very large population of heart disease patients, transmitting the data via the Internet, and remotely storing the data in a database (for example, the so-called Cloud) to build the knowledge for so-called Big Data that may correspond to a variety of heart diseases and their treatments. The Big Data can be collected and used by many different entities, including but not limited to medical researchers, pharmaceutical researchers, medical device researchers, and artificial intelligence systems. The accessing and processing of such data by artificial intelligence from locations far remote from patients enable conditions of potentially millions of patients to be safely and quickly assessed and treated than would be possible if relying solely on human support or intervention. The system can then notify a patient and their medical caregiver, such as via email, text messages, a dedicated communication device, etc., concerning the patient's condition and the status of their disease, and identify in what manner the patient's treatment may be tailored, including medications, medical devices implanted in or otherwise used by the patient, lifestyle, and combinations thereof. The system may also recognize and notify the patient and caregiver regarding the early onset of a potentially dangerous condition(s). If the patient's condition and status of their disease are common or otherwise within the knowledge of the system through available Big Data, tailored treatments may be prescribed and in some situations may even be administered solely by the artificial intelligence capability of the system, for example, by modifying medication levels, adjusting the operating parameters of a medical device, scheduling a physician office visit, contacting an ambulance or other first responder, scheduling a consultation with a specialist, etc. In this manner, the system alone may be able to autonomously manage known (predictable) medical conditions and prescribe safe and effective treatments for certain complicated conditions.

On the other hand, the artificial intelligence of the system is also capable of recognizing situations in which the knowledge stored in the database of the system is insufficient to prescribe or administer tailored treatments solely based on the artificial intelligence capability of the system, in which case the artificial intelligence notifies (via the Internet) at least one member of the patient's medical team that human judgment is required to prescribe an appropriate individualized course of medical action in response to data obtained from the patient. For example, if the system detects an unknown or unpredictable condition, the system may request assistance from a designated medical center. The request is then routed to a medical manager, who uses their expertise, experience, and available resources to propose an appropriate individualized course of medical action, which may be a change the patient's medication, a change in the operation of a medical device (for example, a pacemaker, ventricular assist device (VAD), etc.), scheduling a physician office visit, contacting an ambulance or other first responder, arrange a consultation with a specialist, or in some situations no treatment modification. The medical manager role may be performed by various medical staff members, including a nurse or technician who may seek the assistance of a specialist if a higher level of expertise is required.

The process described above simultaneously occurs with the collection and storing of information, including the data concerning the patient's medical condition and the prescribed treatment and individualized course of medical action, so that the artificial intelligence of the system learns from the medical condition and the individualized course of medical action prescribed by the caregiver. The system also shares this newly acquired information with the medical center or at minimum the medical manager that prescribed the treatment and the individualized course of medical action. Once sufficient data has been collected concerning an unknown or unpredictable medical condition and a successful treatment for that condition has been established, either statistically by the artificial intelligence or as a result of the expertise of a qualified expert, the artificial intelligence of the system identifies the medical condition as a known and predictable medical condition and identifies the medical condition as such to users of the system. In this manner, this process progressively leads to reduced human assistance with unknown, unpredictable, and/or complicated medical situations. The Big Data collected by the system can be used by researchers from various different fields to assist in this continuous evolution.

The collection of data by the system over time can be greatly accelerated by obtaining data from sensors placed within the bodies of very large populations of patients to continuously or periodically sense and monitor one or more physiological parameters in the bodies of the patients and generate data corresponding to the physiological parameter(s). The sensors are preferably chronically implanted and adapted to continuously or periodically sense and monitor one or more physiological parameters (as a nonlimiting example, pressures sensed by wireless implantable hemodynamic monitors (IHMs)), and collect such information from potentially millions of people over their lifetimes to accumulate significant detailed medical data that has never been available before. Notable implantable sensors for this purpose include, but are not limited to, those disclosed in U.S. Pat. Nos. 8,744,544, 8,715,300, 8,696,693, 8,512,252, 8,322,346, 8,267,863, 8,014,865, 7,860,579, 7,686,762, 7,634,319, 7,615,010, 7,317,951, and 6,968,743. Such data may include various different pressures within the cardiovascular system, including but not limited to left heart filling pressure (LHFP), pulmonary capillary wedge pressure (PCWP), and other pressures within individual chambers of the heart or the within the pulmonary circulation system, including the pulmonary artery. This information further includes data relating to many diseases, for example, cardiac diseases, and is relevant to the onset and progression of heart diseases and medical conditions (as nonlimiting examples, atrial fibrillation, hypotension, hypertension, heart failure decompensation, and heart attack) and the effectiveness of their different treatments. This information, collected to create Big Data, can be used by the system to better apply medical treatments including medication or medical device adjustments, and allow for a better understanding of the etiology of many other chronic disease and how to manage them at much earlier stages than currently possible. The Big Data also can be used to better understand the treatment by edication of different drugs, their combinations, doses and timing; or may allow better selection of drug types and doses for individual patients over time.

While the invention has been described in terms of particular embodiments, it should be apparent that alternatives could be adopted by one skilled in the art. As such, it should be understood that the above detailed description is intended to describe the particular embodiments and certain but not necessarily all features and aspects thereof, and to identify certain but not necessarily all alternatives to the embodiments and described features and aspects. As a nonlimiting example, the invention encompasses additional or alternative embodiments in which one or more features or aspects of the disclosed embodiments could be eliminated or two or more features or aspects of different embodiments could be combined. Accordingly, it should be understood that the invention is not necessarily limited to any embodiment described herein or illustrated in the drawing, and the phraseology and terminology employed above are for the purpose of describing the disclosed embodiment and do not necessarily serve as limitations to the scope of the invention. Therefore, the scope of the invention is to be limited only by the following claims.

1. A method of managing at least one chronic disease in a population of patients by collecting information from the population of patients and prescribing courses of medical action based on collaboration between human beings and artificial intelligence (AI), the method comprising:
placing sensors within the human bodies of the population of patients to continuously or periodically sense and monitor at least one physiological parameter in the human bodies and generate data corresponding to the physiological parameter;
transmitting via the Internet the data corresponding to the physiological parameter in the population of patients and storing the data in a database; and
using artificial intelligence to prescribe a course of medical action in an individual patient of the population of patients if the data corresponding to the physiological parameter in the patient indicates a medical condition that is within the database and known by the artificial intelligence, or using the artificial intelligence to request an individualized course of medical action from a medical provider if the data corresponding to the physiological parameter in the patient indicates a medical condition not within the database or not known by the artificial intelligence.

2. The method of claim 1, wherein the sensors comprise implantable hemodynamic monitor sensors and are chronically implanted in the population of patients, and the method comprises generating, transmitting, and storing the data over the lifetimes of the population of patients.

3. The method of claim 1, wherein the course of medical action and the individualized course of medical action comprise one or more of modifying a medication level for the patient, adjusting an operating parameter of a medical device used by the patient, scheduling a physician office visit, contacting a first responder, and scheduling a consultation with a specialist.

4. The method of claim 1, wherein the chronic disease is a heart disease.

5. The method of claim 1, wherein the physiological parameter is a pressure within the cardiovascular systems of the population of patients.

6. The method of claim 1, wherein the physiological parameter is left heart filling pressure (LHFP), pulmonary artery (PA) pressure, pulmonary capillary wedge pressure (PCWP), a pressure within an individual chamber of the heart, or a pressure within the pulmonary circulation system.

7. The method of claim 21, further comprising the artificial intelligence sharing with the medical provider the medical condition and the individualized course of medical action that was learned by the artificial intelligence and stored in the database.

8. The method of claim 1, wherein the medical condition that is not within the database or not known by the artificial intelligence is an unknown or unpredictable medical condition, the unknown or unpredictable medical condition and the individualized course of medical action therefor only become known by the artificial intelligence once sufficient data have been collected concerning the unknown or unpredictable medical condition and a successful treatment for the medical condition has been established by the medical provider or statistically established by the artificial intelligence, and thereafter the system identifies the unknown or unpredictable medical condition as a known or predictable medical condition that does not require obtaining the individualized course of medical action from the medical provider.

9. The method of claim 1, further comprising collecting the data corresponding to the physiological parameter of the population of patients to create Big Data.

10. The method of claim 9, further comprising using the Big Data to predict onset of the chronic disease or the medical condition.

11. The method of claim 10, wherein the chronic disease or the medical condition is atrial fibrillation, hypotension, hypertension, heart failure decompensation, or heart attack.

12. The method of claim 9, further comprising using the Big Data to apply the individualized course of medical action.

13. The method of claim 12, wherein the individualized course of medical action comprises modifying a medication level for the patient or adjusting an operating parameter of a medical device used by the patient.

14. The method of claim 9, further comprising using the Big Data to understand the etiology of the chronic disease and prevention and treatments thereof.

15. A system for managing at least one chronic disease in a population of patients by collecting information from the population of patients and prescribing courses of medical action based on collaboration between human beings and artificial intelligence (AI), the system comprising:
sensors placed within the human bodies of the population of patients to continuously or periodically sense and monitor at least one physiological parameter in the human bodies and generate data corresponding to the physiological parameter; and
artificial intelligence that prescribes a course of medical action in an individual patient of the population of patients if the data corresponding to the physiological parameter in the patient indicates a medical condition that is within the database and known by the artificial intelligence, and requests an individualized course of medical action from a medical provider if the data corresponding to the physiological parameter in the patient indicates a medical condition not within the database or known by the artificial intelligence.

16. The system of claim 15, wherein the sensors comprise implantable hemodynamic monitor sensors and that have been chronically implanted in the population of patients to generate, transmit, and store the data over the lifetimes of the population of patients.

17. The system of claim 15, wherein the course of medical action and the individualized course of medical action comprise one or more of modifying a medication level for the patient, adjusting an operating parameter of a medical device used by the patient, scheduling a physician office visit, contacting a first responder, and scheduling a consultation with a specialist.

18. The system of claim 15, wherein the chronic disease is a heart disease.

19. The system of claim 15, wherein the physiological parameter is a pressure within the cardiovascular systems of the population of patients.

20. The system of claim 15, wherein the physiological parameter is left heart filling pressure (LHFP), pulmonary artery (PA) pressure, pulmonary capillary wedge pressure (PCWP), a pressure within an individual chamber of the heart, or a pressure within the pulmonary circulation system.

21. The method of claim 1, the method further comprising storing the medical condition and the individualized course of medical action prescribed by the medical provider in the database so that the artificial intelligence learns from the medical provider and the medical condition and the individualized course of medical action become known by the artificial intelligence.

22. The system of claim 15, wherein the system stores the medical condition and the individualized course of medical action prescribed by the medical provider in the database so that the artificial intelligence learns from the medical provider and the medical condition and the individualized course of medical action become known by the artificial intelligence.
